(19) United States
(12) Patent Application Publication
(10) Pub. No.: US 2010/0249004 A1
(43) Pub. Date: Sep. 30, 2010

(54) WASHING AND CONDITIONING COSMETIC COMPOSITION COMPRISING FOUR SURFACTANTS AND A NON-SILICONE

(76) Inventor: Géraldine FACK, Levallois-Perret (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 12/644,634

(22) Filed: Dec. 22, 2009

Related U.S. Application Data
(60) Provisional application No. 61/148,186, filed on Jan. 29, 2009.
(60) Provisional application No. 61/149,099, filed on Feb. 2, 2009.

(30) Foreign Application Priority Data
Dec. 22, 2008 (FR) 0858963
Dec. 22, 2008 (FR) 0858965

(51) Int. Cl. A61K 8/49; C11D 17/00; A61Q 5/02; A61Q 5/12

(52) U.S. Cl. 510/124

(57) ABSTRACT

The present disclosure relates to detergent cosmetic compositions for keratin fibers, for example human keratin fibers such as the hair, comprising at least one anionic surfactant (A) comprising in its structure at least one group chosen from sulfate, sulfonate and phosphate groups, at least one carboxylic alkyl ether anionic surfactant (B) other than the at least one anionic surfactant (A), at least one surfactant chosen from amphoteric and zwitterionic surfactants (C), at least one alkyl(poly)glycoside nonionic surfactant (D), at least one non-silicone fatty substance (E), and optionally at least one cationic polymer (F), wherein the weight ratio of the amount of the at least one anionic surfactant (A) to the amount of the at least one nonionic surfactant (D) has a value ranging from 0.5 to 1.

This application claims benefit of U.S. Provisional Application Nos. 61/148,186, filed Jan. 29, 2009, and 61/149,099, filed Feb. 2, 2009. This application also claims benefit of priority under 35 U.S.C. §119 to French Patent Application Nos. 0858963 and 0858965, filed Dec. 22, 2008.

The present disclosure relates to novel compositions for washing and conditioning keratin materials, for example, human keratin fibers such as the hair, comprising at least one solid fatty substance. Also disclosed herein is the use of these compositions, for example as a conditioning shampoo, for treating and washing the keratin materials, such as keratin fibers.

It is known to use conditioning shampoos, for example, conditioning shampoos where a washing base is combined with a hydrophilic conditioning agent, such as a cationic or amphoteric polymer, and an insoluble conditioning agent, such as silicone, a natural or synthetic oil, a fatty substance, or mixtures thereof.

Insoluble conditioning agents, for instance, fatty substances, are known and used for improving the disentangling and softness of wet and dried hair.

However, their use may be greatly limited:

firstly due to the difficulties of stabilization in detergent compositions, if the level of the working qualities is to be maintained (stability, latherability, initiation of lathering),

secondly due to the cosmetic defects in terms of heaviness, lankness and regreasing that may be associated with coarse or heterogeneous dispersions in these same detergent compositions.

Thus, there is still a need for detergent compositions comprising fatty compounds, for example finely dispersed or even dissolved fatty compounds that can have high cosmetic performance qualities in terms of disentangling and smoothing, without making the hair lank or heavy, and while maintaining the level of the working qualities.

The present inventors have discovered that it can be possible to formulate such detergent compositions by combining a specific surfactant base with four components, and at least one non-silicone fatty substance, optionally in the presence of at least one cationic polymer.

One aspect of the present disclosure is a composition for washing and conditioning keratin materials, for example keratin fibers, comprising, in a cosmetically acceptable medium, at least four different surfactants defined below, at least one non-silicone fatty substance, and optionally at least one cationic polymer.

The compositions disclosed herein can make it possible to give excellent cosmetic properties on both dry and wet hair, for instance in terms of disentangling, suppleness, smoothing, sheen and manageability of the hair.

As disclosed herein, a species is termed “anionic” when it contains at least one permanent negative charge or when it may be ionized into a negatively charged species, under the conditions in which the compositions of the disclosure are used (for example, the medium and the pH), not comprising any cationic charge.

Similarly, a species is termed “cationic” when it contains at least one positive charge or when it may be ionized into a positively charged species, under the conditions in which the compositions of the disclosure are used (for example, the medium and the pH), not comprising any anionic charge.

A species is termed “nonionic” when it is neither cationic nor anionic nor comprises any cationic or anionic groups.

As used herein, “cosmetically acceptable” and “physiologically acceptable” mean compatible with use on the body of a living being, such as the human body, for instance, the scalp and the hair.

The following terms may be used herein:

“surfactant (A)” or “compound (A)” is understood to mean an anionic surfactant comprising in its structure at least one sulfate and/or sulfonate and/or phosphate group;

“surfactant (B)” or “compound (B)” means a carboxylic alkyl ether anionic surfactant other than the surfactant (A);

“surfactant (C)” or “compound (C)” means an amphoteric and/or zwitterionic surfactant;

“surfactant (D)” or “compound (D)” means an alkyl(poly)glycoside nonionic surfactant;

“compound (E)” means a mineral or organic non-silicone fatty substance; and

“polymer (F)” or “compound (F)” means a cationic polymer.

As defined according to the present disclosure, compounds (A), (B), (C), (D),

(E) and (F) are different from each other.

Unless otherwise indicated, each of the compounds, optional or otherwise, used or envisaged in the context of the present disclosure may be present alone or as a mixture.

One aspect of the present disclosure is a cosmetic composition for washing and conditioning keratin materials, for example human keratin fibers such as the hair, comprising:

at least one anionic surfactant (A) comprising in its structure at least one entity chosen from sulfate, sulfonate, and phosphate groups,

at least one carboxylic alkyl ether anionic surfactant (B) other than the at least one anionic surfactant mentioned in (A),

at least one surfactant chosen from amphoteric and zwitterionic surfactants (C),

at least one alkyl(poly)glycoside nonionic surfactant (D),

at least one non-silicone fatty substance (E),

and optionally at least one cationic polymer (F), the weight ratio of the amount of the at least one anionic surfactant (A) to the amount of the at least one nonionic surfactant (D) ranging from 0.5 to 1.

Anionic Surfactant(s) (A) Comprising in their Structure at Least One Sulfate and/or Sulfonate and/or Phosphate Group

According to at least one embodiment of the present disclosure, the at least one surfactant (A) does not comprise in its structure any carboxylic groups (COOH) or carboxylic groups in salt form (COO⁻).

The at least one surfactant (A) may be oxyethylenated and/or oxypropylenated. The total average number of ethylene oxide (EO) and/or propylene oxide (PO) groups may then range from 2 to 50, such as from 2 to 10.

Non-limiting examples of the at least one surfactant (A) that may be used include alkyl sulfates, alkylamido sulfates, alkyl ether sulfates, alkylamido ether sulfates, alkylaryl ether sulfates, alkyl ether sulfosuccinates, acyl isethionates and methyl acyl taurates, and salts thereof; the alkyl or acyl group of all these various compounds comprising, for example from 8 to 24 carbon atoms, and the aryl group for instance denoting a phenyl or benzyl group.

In some embodiments, the at least one surfactant (A) is chosen from sulfated anionic surfactants, such as C₈-C₁₄ alkyl ether sulfates, for instance C₁₂-C₁₄ alkyl ether sulfates.

In some embodiments, the at least one surfactant (A) is present in the form of a salt, for example chosen from alkali metal salts, such as a sodium salts, ammonium salts, amine salts, including amino alcohol salts, and magnesium salts. These salts may comprise, for instance, from 2 to 5 ethylene oxide groups. In at least one embodiment, the at least one surfactant (A) is chosen from sodium, triethanolamine, magnesium or ammonium (C₁₂-C₁₄)alkyl sulfates and/or sodium, ammonium and magnesium (C₁₂-C₁₄)alkyl ether sulfates, which are oxyethylenated, for example with 2.2 mol of ethylene oxide. In some embodiments, the at least one surfactant (A) is chosen from sodium, ammonium and magnesium (C₁₂-C₁₄)alkyl ether sulfates oxyethylenated with 2.2 mol of ethylene oxide, as sold, for instance, under the name TEXAPON N702 by the company Cognis.

According to at least one embodiment in the present disclosure, the at least one anionic surfactant (A) is chosen from sodium and ammonium lauryl ether sulfates.

The at least one surfactant (A) is present in an amount ranging from 1% to 50% by weight, for example ranging from 2% to 25% by weight, such as ranging from 3% to 20% by weight relative to the total weight of the composition.

### Carboxylic Alkyl Ether Anionic Surfactant(s) (B)

The at least one carboxylic alkyl ether anionic surfactant (B) in some embodiments can comprise a C₆-C₂₄ alkyl chain.

For example, the at least one surfactant (B) may be chosen from:

(C₆-C₂₄)alkyl ether carboxylic acids,

(C₆-C₂₄)alkylaryl ether carboxylic acids,

(C₆-C₂₄)alkylamido ether carboxylic acids, and

salts thereof.

The at least one surfactant (B) disclosed herein may be oxyalkylenated, for instance oxyethylenated and/or oxypropylenated. In some embodiments, the total average number of alkylene oxide groups ranges from 2 to 50, for example from 2 to 24, such as from 2 to 15.

When the at least one surfactant (B) is oxyalkylenated, it comprises from 2 to 50 alkylene oxide groups, such as from 2 to 50 ethylene oxide (EO) groups.

According to at least one embodiment of the present disclosure, the at least one surfactant (B) is neutralized with at least one salt. Non-limiting examples of such salts include alkali metal salts, for instance sodium salts, ammonium salts, amine salts including amino alcohol salts such as triethanolamine or monoethanolamine salts, and magnesium salts.

In at least one embodiment, the polyethoxylated carboxylic anionic surfactants are chosen from those of formula (I):

R₁(OC₂H₄)ₙOCH₂COOA (I)

wherein:

R₁ is chosen from linear and branched C₈-C₂₂ alkyl groups and linear and branched C₈-C₂₂ alkenyl, (C₈-C₉)alkylphenyl groups, and R₂CONH—CH₂—CH₂— wherein R₂ is chosen from linear and branched C₁₁-C₂₁ alkyl groups and linear and branched C₁₁-C₂₁ alkenyl groups,

n is an integer or decimal number (average value) ranging from 2 to 24, such as from 2 to 10,

A is chosen from H, NH₄, Na, K, Li, Mg and monoethanolamine and triethanolamine residues. As discussed above, mixtures of compounds of formula (I) may be used, for example mixtures wherein the groups R₁ are different.

According to at least one embodiment, R₁ is chosen from C₁₂-C₁₄ alkyl, cocoyl, oleyl, nonylphenyl and octylphenyl groups; A is chosen from a hydrogen atom and a sodium atom; and n ranges from 2 to 20, such as from 2 to 10.

In some embodiments, R₁ is a C₁₂ alkyl group; A is chosen from a hydrogen atom and a sodium atom; and n ranges from 2 to 10.

Among the commercial products that may be used according to the present disclosure, include, but are not limited to the products sold by the company Chem Y under the names:

AKYPO® NP 70 (R₁=nonylphenyl, n=7, A=H)

AKYPO® NP 40 (R₁=nonylphenyl, n=4, A=H)

AKYPO® OP 40 (R₁=octylphenyl, n=4, A=H)

AKYPO® OP 80 (R₁=octylphenyl, n=8, A=H)

AKYPO® OP 190 (R₁=octylphenyl, n=19, A=H)

AKYPO® RLM 38 (R₁=(C₁₂-C₁₄)alkyl, n=4, A=H)

AKYPO® RLM 38 NV (R₁═(C₁₂-C₁₄)alkyl, n=4, A=Na)

AKYPO® RLM CA 45 (R₁=(C₁₂-C₁₄)alkyl, n=4,5, A=H)

AKYPO® RLM 45 NV (R₁═(C₁₂-C₁₄)alkyl, n=4.5, A=Na)

AKYPO® RLM 100 (R₁=(C₁₂-C₁₄)alkyl, n=10, A=H)

AKYPO® RLM 100 NV (R₁═(C₁₂-C₁₄)alkyl, n=10, A=Na)

AKYPO® RLM 130 (R₁=(C₁₂-C₁₄)alkyl, n=13, A=H)

AKYPO® RLM 160 NV (R₁═(C n=16, A=Na)

or by the company Sandoz under the names:

SANDOPAN DTC-Acid (R₁═(C₁₃)alkyl, n=6, A=H)

SANDOPAN DTC (R₁=(C₁₃)alkyl, n=6, A=Na)

SANDOPAN LS 24 (R₁=(C₁₂-C₁₄)alkyl, n=12, A=Na)

SANDOPAN JA 36 (R₁=(C₁₃)alkyl, n=18, A=H),

and the products sold under the following names:

AKYPO® RLM 45 (INCI: Laureth-5 carboxylic acid)

AKYPO® RLM 100

AKYPO® RLM 38.

According to at least one embodiment, the at least one carboxylic anionic surfactant (B) is chosen from sodium lauryl ether carboxylates.

The at least one surfactant (B) is present in an amount ranging from 0.5% to 15% by weight, for instance from 1 to 10% by weight, such as from 1.5% to 8% by weight relative to the total weight of the composition.

Amphoteric and/or Zwitterionic Surfactant(s) (C)

The at least one surfactant chosen from amphoteric and zwitterionic surfactants that may be used herein may be chosen from, by way of non-limiting manner, aliphatic secondary and tertiary amine derivatives, wherein the aliphatic substituents(s) on the secondary or tertiary amine function are chosen from linear and branched chains comprising from 8 to 22 carbon atoms and containing at least one hydro-solubilizing anionic group, for instance a carboxylate, sulfonate, sulfate, phosphate and/or phosphonate group; and/or betaines.

Among the amine derivatives that may be used in the context of the present disclosure, include, but are not limited to the products described in U.S. Pat. No. 2,528,378 and U.S. Pat. No. 2,781,354 and corresponding to one of the structures of formulae (II) and (III) below:

formula (II): R₂—CONHCH₂CH₂—N⁺(R₃)(R₄)(CH₂COO⁻) (II)

wherein:
R₂—CO is chosen from C₆-C₂₄ acyl groups; for example the C₆-C₂₄ acyl part corresponding to one of the fatty acids of formula R₂—COON present in hydrolysed coconut oil, an octanoyl group, a decanoyl group or a dodecanoyl group,
R₃ is a β-hydroxyethyl group,

R₄ is a carboxymethyl group;

formula (III): R₂—CONHCH₂CH₂—N(B)(C) (III)

wherein:

B is —CH₂CH₂OX′,

C is —(CH₂)ₙ—Y′, with z=1 or 2,

X′ is chosen from —CH₂CH₂—COOH and a hydrogen atom,

Y′ is chosen from —COOH and —CH₂—CHOH—SO₃H,

R_{2′}, —CO is chosen from C₆-C₂₄ acyl groups; for example, the C₆-C₂₄ acyl part corresponding to one of the fatty acids of formula R₂—COOH present in hydrolysed coconut oil or linseed oil, an octanoyl group, a decanoyl group, a dodecanoyl group, a stearoyl group, an isostearoyl group or an oleoyl group.

These compounds are classified in the CTFA dictionary, under the names disodium cocoamphodiacetate, disodium lauroamphodiacetate, disodium caprylamphodiacetate, disodium capryloamphodiacetate, disodium cocoamphodipropionate, disodium lauroamphodipropionate, disodium caprylampho-dipropionate, disodium capryloamphodipropionate, lauroamphodipropionic acid and cocoamphodipropionic acid.

A non-limiting example that may be mentioned according to the present disclosure is disodium cocoamphodiacetate, sold under the trade name MIRANOL® C2M Concentrate by the company Rhodia Chimie.

Among the betaines that may be used herein, non-limiting mention may be made of (C₈-C₂₀)alkyl betaines, sulfobetaines, (C₈-C₂₀)alkylamido(C₁-C₆)alkyl betaines and (C₈-C₂₀)alkylamido(C₁-C₆)alkyl sulfobetaines, optionally hydroxylated, and mixtures thereof.

In at least one embodiment, the at least one amphoteric and/or zwitterionic surfactant that may be used herein include, but are not limited to (C₈-C₂₀)alkyl betaines, such as the cocobetaine sold by the company Cognis under the name DEHYTON AB 30 as an aqueous solution containing 30% by weight of Active Material (AM) relative to the total weight of the solution; (C₈-C₂₀)alkylamido(C₁-C₆)alkyl betaines, for instance cocamidopropylbetaine such as the product sold under the name TEGOBETAINE® F50 by the company Goldschmidt.

In at least one embodiment, the at least one amphoteric and/or zwitterionic surfactant (C) is chosen from betaines.

In another embodiment, the at least one amphoteric and/or zwitterionic surfactant (C), is chosen from cocamidopropylbetaine and cocobetaine.

The at least one amphoteric and/or zwitterionic surfactant (C) is present in an amount ranging from 0.1% to 20% by weight, for example from 1% to 15% by weight, such as from 2% to 10% by weight relative to the total weight of the composition.

### Alkyl(poly)glycoside Nonionic Surfactants) (D)

As used herein, the term “alkyl(poly)glycoside” is understood to mean an alkylpolyglycoside or an alkylmonoglycoside, or an alkylglycoside, which may be alkoxylated with at least one alkylene oxide group, such as a C₂-C₄ alkylene oxide group.

The at least one alkyl(poly)glycoside nonionic surfactant (D) used, in accordance with the present disclosure may be represented by formula (IV) below:

R₁O—(R₂O)ₜ-(G)ᵥ (IV)

wherein:

R₁ is chosen from linear and branched, saturated and unsaturated alkyl groups, comprising from 8 to 24 carbon atoms, or an alkyl phenyl group wherein the linear or branched alkyl radical comprises from 8 to 24 carbon atoms,

R₂ is an alkylene group comprising from 2 to 4 carbon atoms,

G is a saccharide unit comprising 5 or 6 carbon atoms,

t is a value ranging from 0 to 10, such as from 0 to 4, and

v is a value ranging from 1 to 15.

In at least one embodiment, the at least one alkyl(poly)glycoside nonionic surfactant (D) corresponds to formula (IV) wherein:

R₁ is chosen from linear and branched, saturated and unsaturated alkyl groups comprising from 8 to 18 carbon atoms,

G is chosen from glucose, fructose and galactose, and in at least one embodiment glucose,

t is a value ranging from 0 to 3, and in one embodiment 0, and R₂ and v are as defined previously.

The degree of polymerization of the at least one alkyl(poly)glycoside nonionic surfactant (D) as represented, for example, by the index v in formula (IV) ranges on average from 1 to 15, such as from 1 to 4. In at least one embodiment, this degree of polymerization ranges from 1 to 2, such as from 1.1 to 1.5, on average.

The glycoside bonds between the saccharide units are 1,6- or 1,4-bonds; such as 1,4-bonds.

The compounds of formula (IV) that may be used in accordance with the present disclosure include, by way of non-limiting example the products sold by the company Cognis under the names PLANTAREN® (600 CS/U, 1200 and 2000) or PLANTACARE® (818, 1200 and 2000). It is also possible to use the products sold by the company SEPPIC under the names TRITON CG 110 (or ORAMIX CG 110) and TRITON CG 312 (or ORAMIX® NS 10), the products sold by the company BASF under the name LUTENSOL GD 70, or those sold by the company Chem Y under the name AG10 LK.

It is also possible, for example, to use the 1,4-(C₈-C₁₆)alkylpolyglucoside as an aqueous solution at 53% by weight relative to the total weight of the solution, sold by Cognis under the reference PLANTACARE® 818 UP.

According to at least one embodiment in the present disclosure, the at least one alkyl(poly)glycoside nonionic surfactant (D) is chosen from cocoylpolyglucoside (INCI: Coco glucoside) sold by Cognis under the reference PLANTACARE® 818.

The at least one alkyl(poly)glycoside nonionic surfactant (D) is present in an amount ranging from 0.1% to 20% by weight, such as from 1% to 15% by weight, for instance from 2% to 10% by weight relative to the total weight of the composition.

The minimum amount of anionic, amphoteric and/or zwitterionic surfactants, and nonionic surfactants, is that which is sufficient to give the composition satisfactory latherability and/or detergent power.

Thus, according to the present disclosure, the total amount of anionic, amphoteric and/or zwitterionic surfactants, and nonionic surfactants, may be present in an amount ranging from 4% to 50% by weight, for example from 6% to 35% by weight, such as from 8% to 25% by weight, relative to the total weight of the composition.

The weight ratio of the amount of the at least one surfactant (A) to the amount of the at least one surfactant (B) ranges from 0.1 to 10, for example, from 0.5 to 5.

The weight ratio of the amount of the at least one surfactant (A) to the amount of the at least one surfactant (C) has a value ranging from 0.1 to 10, for instance from 0.5 to 5, such as from 0.7 to 2.

The weight ratio of the amount of the at least one anionic surfactant (B) to the amount of the at least one surfactant (C) has a value ranging from 0.1 to 10, for example from 0.2 to 5.

### Non-Silicone Fatty Substance(s) (E)

The at least one non-silicone fatty substance (E) used according to the present disclosure may be solid or liquid.

As used herein, “non-silicone solid fatty substance” means an organic compound that has in its structure at least one carbon chain comprising at least six carbon atoms, which is solid and insoluble in water at room temperature (25° C.) and at atmospheric pressure, with a melting point of greater than or equal to 35° C. and/or a viscosity, at a temperature of 40° C. and at a shear rate of 1 s⁻¹, of greater than or equal to 1 Pa·s.

The term “insoluble in water” is understood to mean a compound that has a solubility in water of less than 1% by weight, such as less than 0.5% by weight. The solid fatty substance is, in at least one embodiment, soluble at room temperature (25° C.) and at atmospheric pressure in at least one organic solvent (for example ethanol, chloroform or benzene) to at least 1% by weight.

The at least one non-silicone solid fatty substance (E) according to the disclosure may be crystalline, amorphous or pasty.

The melting point ranges, for example, from 35° C. to 250° C., such as from 40 to 150° C. These solid fatty substances have a viscosity, at a temperature of 40° C. and at a shear rate of 1 s⁻¹, ranging from 1 Pa·s to 1,000,000 Pa·s, for instance, from 10 to 1,000 Pa·s.

The viscosity measurements may be taken at a temperature of about 40° C., on a Carri-Med CSL 2-500 viscometer.

The melting points may be measured by DSC or on a Köfler block. The melting point may be measured by differential colorimetric analysis (DSC) with a temperature rise of 10° C. per minute. The melting point is then the temperature corresponding to the top of the melting endotherm peak obtained during the measurement.

The at least one non-silicone solid fatty substance (E) with a melting point of greater than or equal to 35° C. may be chosen from, by way of non-limiting example, fatty alcohols, fatty esters, fatty acids, waxes without an amide function of animal, plant, mineral or marine origin, and waxes with amide function(s) such as ceramides and ceramide derivatives.

The fatty alcohols according to the present disclosure are in at least one embodiment, linear and saturated, and comprise from 12 to 40 carbon atoms.

The fatty alcohols in one embodiment have the structure ROH, wherein R is chosen from C₁₂-C₂₄ alkyl groups, and is optionally substituted with at least one hydroxyl group or is, in another embodiment, unsubstituted.

Non-limiting examples that may be mentioned herein include myristyl alcohol, cetyl alcohol, stearyl alcohol and behenyl alcohol, and mixtures thereof.

According to some embodiments of the present disclosure, the fatty alcohol may be a mixture of fatty alcohols, which means that several species of fatty alcohol may coexist in a commercial product, in the form of a mixture.

The fatty alcohols of the disclosure are non-oxyalkylenated and/or non-glycerolated. These fatty alcohols may be constituents of animal or plant waxes.

The fatty esters herein are esters comprising at least 10 carbon atoms, and in one embodiment, esters of a carboxylic acid comprising at least 10 carbon atoms and of a monoalcohol or a polyol. The fatty esters according to the present disclosure may be mono-, di- or triesters.

The carboxylic acids may comprise from 10 to 30 carbon atoms, such as from 12 to 24 carbon atoms. The alcohols may comprise from 10 to 30 carbon atoms, such as from 12 to 24 carbon atoms. In at least one embodiment of the present disclosure, the solid fatty esters disclosed herein are esters of a monocarboxylic fatty acid comprising at least 10 carbon atoms and of a monoalcohol comprising at least 10 carbon atoms.

Non-limiting examples of esters that may be mentioned include cetyl myristate, myristyl myristate, palmityl palmitate, stearyl palmitate, palmityl stearate and stearyl stearate, and mixtures thereof.

The fatty esters may be constituents of animal or plant waxes.

The fatty acids, in at least one embodiment, comprise from 10 to 30 carbon atoms, such as from 12 to 24 carbon atoms. The fatty acids disclosed herein, include but are not limited to stearic acid, behenic acid and lauric acid, and mixtures thereof.

As used herein, a wax is a lipophilic compound, which is solid at room temperature (about 25° C.), with a reversible solid/liquid change of state, having a melting point greater than about 40° C., which may be up to 200° C., and having in the solid state anisotropic crystal organization. In general, the size of the wax crystals is such that the crystals diffract and/or scatter light, giving the composition that comprises them a more or less opaque cloudy appearance.

Non-limiting examples of waxes without an amide function that may be used herein include, but are not limited waxes of animal origin such as spermaceti, lanolin wax and lanolin derivatives, beeswaxes and modified beeswaxes (cerabellina); plant waxes such as carnauba wax, candelilla wax, esparto grass wax, ouricury wax, Japan wax, olive wax, rice wax, hydrogenated jojoba wax or the absolute waxes of flowers such as the essential wax of blackcurrant blossom sold by the company Bertin (France), shea butter, cocoa butter, cork fibre wax or sugarcane wax; mineral waxes, for example paraffin wax, petroleum jelly wax, lignite wax, microcrystalline waxes and ozokerites; other waxes or waxy starting materials that may be used according to the present disclosure include, by way of non-limiting mention marine waxes, such as the product sold by the company Sophim under the reference M82, and mixtures thereof.

As used herein, the term “ceramides or ceramide derivative” means a natural or synthetic compound chosen from ceramides, glycoceramides, pseudoceramides and neoceramides.

Such compounds are described, for example, in German Patent Application Nos. DE 4 424 530, DE 4 424 533, DE 4 402 929, and DE 4 420 736, International Patent Application Publication Nos. WO 95/23807, WO 95/16665, WO 94/07844, WO 94/24097 and WO 94/10131, French Patent Application No. FR-2 673 179, and European Patent Application Nos. EP-A-0 227 994, and EP-A-0 646 572.

The waxes containing amide function(s), including ceramides or ceramide derivatives, which may be used according to the present disclosure correspond, for example, to the general formula (V) below:

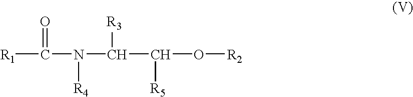

wherein:

R₁ is chosen from:

linear and branched, saturated and unsaturated C₁-C₅₀ hydrocarbon based groups, for example C₅-C₅₀ hydrocarbon-based groups, optionally substituted with at least one hydroxyl group optionally esterified with an acid R₇COOH, wherein R₇ is chosen from saturated and unsaturated, linear and branched, optionally mono- or polyhydroxylated C₁-C₃₅ hydrocarbon-based groups, it being possible for the hydroxyl(s) of the group R₇ to be esterified with a saturated or unsaturated, linear or branched, optionally mono- or polyhydroxylated C₁-C₃₅ fatty acid;

R″—(NR—CO)—R′, wherein R is chosen from a hydrogen atom and mono- or polyhydroxylated, such as monohydroxylated, C₁-C₂₀ hydrocarbon-based groups, R′ and R″ are hydrocarbon-based groups wherein the sum of the carbon atoms ranges from 9 to 30, and R′ is a divalent group;

R₈—O—CO—(CH₂)ₚ, wherein R₈ is chosen from C₁-C₂₀ hydrocarbon-based groups, and p is an integer ranging from 1 to 12;

R₂ is chosen from a hydrogen atom, a group of saccharide type, for example a (glycosyl)ₙ, (galactosyl)ₘ or sulfogalactosyl group, a sulfate or phosphate residue, a phosphorylethylamine group and a phosphorylethylammonium group, wherein n is an integer ranging from 1 to 4 and m is an integer ranging from 1 to 8;
R₃ is chosen from a hydrogen atom and saturated and unsaturated, optionally hydroxylated C₁-C₃₃ hydrocarbon-based groups, it being possible for the hydroxyl(s) to be esterified with an inorganic acid or an acid R₇COOH, R₇ having the same meanings as above, it being possible for the hydroxyl(s) to be etherified with a (glycosyl)ₙ, (galactosyl)ₘ, sulfogalactosyl, phosphorylethylamine or phosphorylethylammonium group wherein n and m are as defined above, it also being possible for R₃ to be substituted with at least one C₁-C₁₄ alkyl radical; and in one embodiment, R₃ is chosen from C₁₅-C₂₆ α-hydroxyalkyl groups, wherein the hydroxyl group is optionally esterified with a C₁₆-C₃₀ α-hydroxy acid;

R₄ is chosen from a hydrogen atom, methyl and ethyl groups, saturated and unsaturated, linear and branched, optionally hydroxylated C₃-C₅₀ hydrocarbon-based groups, —CH₂—CHOH—CH₂—O—R₆ wherein R₆ is chosen from C₁₀-C₂₆ hydrocarbon-based groups and radical R₈—O—CO—(CH₂)ₚ, wherein R₈ is chosen from C₁-C₂₀ hydrocarbon-based groups and p is an integer ranging from 1 to 12;

R₅ is chosen from a hydrogen atom, saturated and unsaturated, linear and branched, optionally mono- or polyhydroxylated C₁-C₃₀ hydrocarbon-based groups, it being possible for the hydroxyl(s) to be etherified with a (glycosyl)ₙ, (galactosyl)ₘ, sulfogalactosyl, phosphorylethylamine or phosphorylethylammonium group wherein n and m are as defined above, with the proviso that when R₃ and R₅ are hydrogen atoms or when R₃ is a hydrogen atom and R₅ is a methyl, then R₄ is not a hydrogen atom or a methyl or ethyl group.

In at least one embodiment, the compounds of formula (V), are chosen from ceramides and/or glycoceramides whose structure is described by Downing in Journal of Lipid Research Vol. 35, 2060-2068, 1994, or those described in French Patent Application No. FR-2 673 179.

Suitable ceramides and ceramide derivatives that may be used herein include the compounds of formula (V) wherein R₁ is chosen from saturated and unsaturated, optionally hydroxylated alkyl groups derived from C₁₄-C₂₂ fatty acids; R₂ is a hydrogen atom; and R₃ is chosen from optionally hydroxylated, linear C₁₁-C₁₇ groups, such as C₁₃-C₁₅ groups.

Non-limiting examples include:
2-N-linoleoylaminooctadecane-1,3-diol,
2-N-oleoylaminooctadecane-1,3-diol,
2-N-palmitoylaminooctadecane-1,3-diol,
2-N-stearoylaminooctadecane-1,3-diol,
2-N-behenoylaminooctadecane-1,3-diol,
2-N-[2-hydroxypalmitoyl]aminooctadecane-1,3-diol,
2-N-stearoylaminooctadecane-1,3,4-triol, such as N-stearoylphytosphingosine,
2-N-palmitoylaminohexadecane-1,3-diol, and
   mixtures thereof.

It is also possible to use the compounds of formula (V) wherein R₁ is chosen from saturated and unsaturated alkyl groups derived from C₁₂-C₂₂ fatty acids; R₂ is a galactosyl or sulfogalactosyl group; and R₃ is chosen from saturated and unsaturated C₁₂-C₂₂ hydrocarbon-based groups, for example a CH(OH)—CH═CH—(CH₂)₁₂—CH₃ group.

By way of non-limiting example, mention may be made of the product comprising a mixture of glycoceramides, sold under the trade name GLYCOCER by the company Waitaki International Biosciences.

Other useful examples include the ceramides and ceramide derivatives whose formulae are represented in European Patent Application No. EP-A-0 227 994 and International Patent Application Publication No. WO 94/07844.

Such compounds are, for example, QUESTAMIDE H (bis(N-hydroxyethyl-N-cetyl)malonamide) sold by the company Quest.

It is also possible to use N-docosanoyl-N-methyl-D-glucamine described in International Patent Application Publication No. WO 94/24097.

In at least one embodiment, the non-silicone solid fatty substance (E) is not a fatty acid.

In another embodiment, the at least one non-silicone solid fatty substance (E) is chosen from, 2-oleamido-1,3-octadecanediol and/or carnauba wax.

As used herein, the term “non-silicone liquid fatty substance” means an organic compound comprising in its structure at least one carbon chain containing at least six carbon atoms and not comprising any silicon atoms in its structure, and which, at room temperature (25° C.) and at atmospheric pressure, is a water-insoluble liquid (i.e. it has a solubility in water of less than 1% by weight, such as less than 0.5% by weight), and is soluble, under the same temperature and pressure conditions, in at least one organic solvent (for example ethanol, chloroform or benzene) to at least 1% by weight.

In one embodiment of the present disclosure, the at least one non-silicone liquid fatty substance (E) is chosen from: liquid fatty esters, for example triglycerides, such as plant oils,

hydrocarbon-based oils,

liquid fatty acids, for instance C₈-C₃₀ liquid fatty acids containing branched carbon chains or bearing at least one unsaturation, such as 1 to 3 unsaturations, and

liquid fatty alcohols, for example C₈-C₃₀ liquid fatty alcohols, containing branched carbon chains or bearing at least one unsaturation, such as 1 to 3 unsaturations.

The liquid fatty esters are liquid esters of carboxylic acids and of alcohols, at least one from among the acid and the alcohol comprising at least 8 carbon atoms, such as comprising from 10 to 40 carbon atoms. The acids and alcohols that are the source of the esters may be linear or branched and saturated or unsaturated (with 1 to 3 unsaturations). The alcohol may be a monoalcohol or a polyol. The acid may be optionally mono- or polyhydroxylated.

Non-limiting examples of fatty acid esters that may be used according to the present disclosure include stearyl octanoate (PURCELLIN® oil), isopropyl myristate, isopropyl palmitate, butyl stearate, hexyl laurate, diisopropyl adipate, isononyl isononanoate, 2-ethylhexyl palmitate, 2-octyldodecyl lactate, isostearyl neopentanoate, tridecyl neopentanoate, isocetyl neopentanoate and isoarachidyl neopentanoate, and mixtures thereof.

The plant oils may be chosen from sweet almond oil, avocado oil, castor oil, olive oil, jojoba oil, sunflower oil, wheatgerm oil, sesame oil, groundnut oil, grape seed oil, soybean oil, rape seed oil, safflower oil, coconut oil, corn oil, hazelnut oil, palm oil, apricot kernel oil, cade oil, liquid jojoba wax and beauty-leaf oil, and mixtures thereof.

The hydrocarbon-based oils are for instance non-alkoxylated alkanes comprising at least 8 carbon atoms.

Hydrocarbon-based oils that may be mentioned, by way of non-limiting example, include mineral oils, such as liquid paraffin and liquid petroleum jelly, isoparaffins such as polyisobutylenes, and polydecenes; and mixtures thereof.

In at least one embodiment, the fatty acids include, but are not limited to C₁₀-C₃₂ fatty acids. In some embodiments, the fatty acids are branched and/or comprise 1 to 3 unsaturations. Mention may be made, for instance of isostearic acid and oleic acid.

The fatty alcohols according to the present disclosure are, in at least one embodiment, branched and/or unsaturated, contain from 12 to 40 carbon atoms, and are non-oxyalkylenated.

The fatty alcohols, for instance can have the structure R—OH, wherein R is chosen from branched C₁₂-C₂₄ alkyl and C₁₂-C₂₄ alkenyl groups, optionally substituted with at least one hydroxyl group. In at least one embodiment, R does not contain any hydroxyl groups.

Non-limiting examples that may be mentioned include coley' alcohol, isocetyl alcohol, isostearyl alcohol, 2-octyl-1-dodecanol and 2-ethylhexyldodecanol, and mixtures thereof.

The fatty alcohols, fatty acids or fatty esters may be mixtures, which means that several species, such as with different chain lengths may coexist in for example, a commercial product, in the form of a mixture.

According to at least one embodiment, the at least one non-silicone liquid fatty substance (E) is chosen from liquid fatty esters and/or hydrocarbon-based oils, for instance plant oils, such as avocado oil, isoparaffins, apricot oil and/or isopropyl myristate.

The at least one non-silicone fatty substance (E) may be present in the composition in an amount ranging from 0.05% to 8% by weight, for instance from 0.1% to 5% by weight, such as from 0.5% to 3% by weight relative to the total weight of the composition.

### Cationic Polymer(s) (F)

When the composition according to the present disclosure comprises at least one cationic polymer (F), at least one cationic polymer (F) may be chosen from all those already known in the art to improve the cosmetic properties of hair treated with detergent compositions, such as those described in European Patent Application No. EP-A-0 337 354 and in French Patent Application Nos. FR-A-2 270 846, FR-A-2 383 660, FR-A-2 598 611, FR-A-2 470 596, FR-A-2 519 863 and FR-A-2 875 503.

According to at least one embodiment, the at least one cationic polymer (F) is chosen from those that contain in their structure units containing primary, secondary, tertiary and/or quaternary amine groups which can either form part of the main polymer chain or which can be carried by a lateral substituent that is directly attached thereto.

Among the cationic polymers, non-limiting mention may be made of polymers of the family of polyamines, polyamino amides and polyquaternary ammoniums. For example, mention may be made of:

(1) Homopolymers or copolymers derived from crosslinked or non-crosslinked acrylic or methacrylic esters or amides and comprising at least one of the units of formula (VI), (VII), (VIII) or (IX) below:

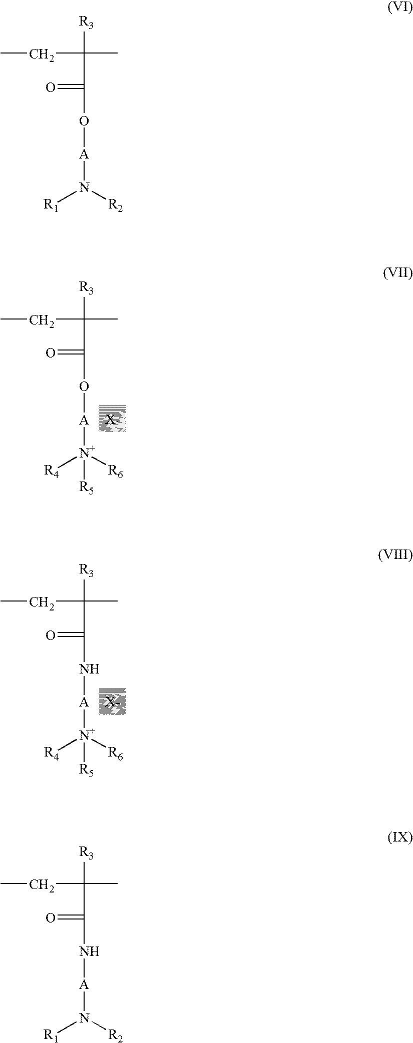

wherein:

R₁ and R₂, which may be identical or different, are each independently chosen from a hydrogen atom and alkyl groups comprising from 1 to 6 carbon atoms, such as methyl and ethyl;

Each instance of R₃, which may be identical or different, is chosen from a hydrogen atom and a CH₃ group;

Each instance of A, which may be identical or different, is chosen from linear and branched alkyl groups comprising from 1 to 6 carbon atoms, such as from 2 or 3 carbon atoms, or a hydroxyalkyl group comprising from 1 to 4 carbon atoms;

Each instance of R₄, R₅ and R₆, which may be identical or different, are chosen from alkyl groups comprising from 1 to 6 carbon atoms or a benzyl group and in some embodiments, an alkyl group containing from 1 to 6 carbon atoms;

X⁻ is an anion derived from an inorganic or organic acid, such as a methosulfate anion or a halide such as chloride or bromide.

Polymers of family (1) can also contain at least one unit derived from comonomers which may be chosen from the family of acrylamides, methacrylamides, diacetone acrylamides, acrylamides and methacrylamides substituted on the nitrogen with lower (C₁-C₄) alkyls, acrylic or methacrylic acids or esters thereof, vinyllactams such as vinylpyrrolidone or vinylcaprolactam, and vinyl esters.

Thus, among these polymers of family (1), non-limiting mention may be made of:

copolymers of acrylamide and of dimethylaminoethyl methacrylate quaternized with dimethyl sulfate or with a dimethyl halide, such as the product sold under the name HERCOFLOC by the company Hercules,

the copolymers of acrylamide and of methacryloyloxyethyltrimethylammonium chloride described, for example, in European Patent Application No. EP-A-080 976 and sold under the name BINA QUAT P 100 by the company Ciba Geigy,

the copolymer of acrylamide and of methacryloyloxyethyltrimethylammonium methosulfate sold under the name RETEN by the company Hercules,

quaternized or non-quaternized vinylpyrrolidone/dialkylaminoalkyl acrylate or methacrylate copolymers, such as the products sold under the name GAFQUAT by the company ISP, such as, GAFQUAT 734 or GAFQUAT 755, or alternatively the products known as Copolymer 845, 958 and 937. These polymers are described in detail in French Patent Nos. 2 077 143 and 2 393 573,

dimethylaminoethyl methacrylate/vinylcaprolactam/vinylpyrrolidone terpolymers, such as the product sold under the name GAFFIX VC 713 by the company ISP,

vinylpyrrolidone/methacrylamidopropyldimethylamine copolymers sold in particular under the name STYLEZE CC 10 by ISP, and

quaternized vinylpyrrolidone/dimethylaminopropylmethacrylamide copolymers such as the product sold under the name “GAFQUAT HS 100” by the company ISP,

crosslinked methacryloyloxy(C₁-C₄)alkyltri(C₁-C₄)alkylammonium salt polymers such as the polymers obtained by homopolymerization of dimethylaminoethyl methacrylate quaternized with methyl chloride, or by copolymerization of acrylamide with dimethylaminoethyl methacrylate quaternized with methyl chloride, the homo- or copolymerization being followed by crosslinking with a compound containing olefinic unsaturation, for example methylenebisacrylamide. A crosslinked acrylamide/methacryloyl-oxyethyltrimethylammonium chloride copolymer (20/80 by weight) in the form of a dispersion containing 50% by weight of said copolymer in mineral oil can be used herein. This dispersion is sold under the name SALCARE® SC 92 by the company Ciba. A crosslinked methacryloyloxyethyltrimethylammonium chloride homopolymer containing about 50% by weight of the homopolymer in mineral oil or in a liquid ester can also be used. These dispersions are sold under the names SALCARE® SC 95 and SALCARE® SC 96 by the company Ciba.

(2) Cationic polysaccharides chosen, for example, from:

a) The cellulose ether derivatives containing quaternary ammonium groups, described in French Patent No. 1 492 597, for example the polymers sold under the names “JR” (JR 400, JR 125 and JR 30M) or “LR” (LR 400, or LR 30M) by the company Union Carbide Corporation. These polymers are also defined in the CTFA dictionary as quaternary ammoniums of hydroxyethylcellulose that has reacted with an epoxide substituted with a trimethylammonium group.

b) Cellulose copolymers or cellulose derivatives grafted with a water-soluble monomer of quaternary ammonium, and described, for example in U.S. Pat. No. 4,131,576, such as hydroxyalkylcelluloses, for instance hydroxymethyl-, hydroxyethyl- or hydroxypropylcelluloses grafted, for example, with a methacryloylethyltrimethylammonium, methacrylamidopropyltrimethylammonium or dimethyldiallylammonium salt.

The commercial products corresponding to this definition are sold, for example under the names CELQUAT L 200 and CELQUAT H 100 by the company National Starch.

c) Guar gums containing cationic trialkylammonium groups. Guar gums modified with a salt (e.g. chloride) of 2,3-epoxypropyltrimethylammonium are used, for example.

Such products are sold, for instance, under the trade names JAGUAR C13 S, JAGUAR C 15, JAGUAR C 17 or JAGUAR C162 by the company Meyhall.

(3) Polymers formed from piperazinyl units and divalent alkylene or hydroxyalkylene radicals containing straight or branched chains, optionally interrupted by oxygen, sulfur or nitrogen atoms or by aromatic or heterocyclic rings, and also the oxidation and/or quaternization products of these polymers. Such polymers are described, for instance, in French Patent Nos. 2 162 025 and 2 280 361.

(4) Water-soluble cationic polyamino amides prepared, for instance by polycondensation of an acidic compound with a polyamine; these polyamino amides can be crosslinked with an epihalohydrin, a diepoxide, a dianhydride, an unsaturated dianhydride, a bis-unsaturated derivative, a bis-halohydrin, a bis-azetidinium, a bis-haloacyldiamine, a bis-alkyl halide or alternatively with an oligomer resulting from the reaction of a difunctional compound which is reactive with a bis-halohydrin, a bis-azetidinium, a bis-haloacyldiamine, a bis-alkyl halide, an epihalohydrin, a diepoxide or a bis-unsaturated derivative; these polyamino amides can be alkylated or, if they contain one or more tertiary amine functions, they can be quaternized. Such polymers are described, for example, in French Patent Nos. 2 252 840 and 2 368 508.

(5) The polyamino amide derivatives resulting from the condensation of polyalkylene polyamines with polycarboxylic acids followed by alkylation with difunctional agents. Mention may be made, for example, of adipic acid/dialkylaminohydroxyalkyldialkylenetriamine polymers wherein the alkyl radical comprise from 1 to 4 carbon atoms, such as methyl, ethyl and propyl. Such polymers are described, for instance in French Patent No. 1 583 363.

Among these derivatives, non-limiting mention may be made, for instance of the adipic acid/dimethylaminohydroxypropyl/diethylenetriamine polymers sold under the name CARTARETINE F, F4 or F8 by the company Sandoz.

(6) The polymers obtained by reaction of a polyalkylene polyamine containing two primary amine groups and at least one secondary amine group with a dicarboxylic acid chosen from diglycolic acid and saturated aliphatic dicarboxylic acids having from 3 to 6 carbon atoms. The molar ratio between the polyalkylene polyamine and the dicarboxylic acid ranges from 0.8:1 to 1.4:1; the polyamino amide resulting therefrom is reacted with epichlorohydrin in a molar ratio of epichlorohydrin relative to the secondary amine group of the polyamino amide ranges from 0.5:1 to 1.8:1. Such polymers are described for example in U.S. Pat. Nos. 3,227,615 and 2,961,347.

Polymers of this type are sold, for instance under the name HERCOSETT 57 by the company Hercules Inc. or alternatively under the name PD 170 or DELSETTE 101 by the company Hercules in the case of the adipic acid/epoxypropyl/diethylenetriamine copolymer.

(7) Cyclopolymers of alkyldiallylamine or of dialkyldiallylammonium, such as the homopolymers or copolymers containing, as main constituent of the chain, units corresponding to formula (Xₐ) or (X_{b}):

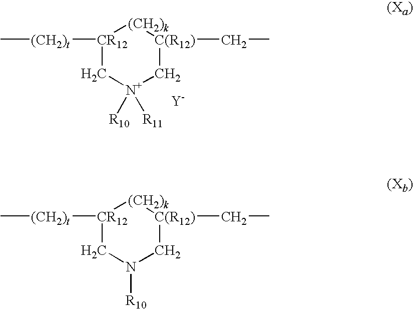

wherein k and t are 0 or 1, the sum k+t is 1; R₁₂ is a hydrogen atom or a methyl radical; R₁₀ and R₁₁, independently of each other, are chosen from alkyl groups comprising from 1 to 6 carbon atoms, hydroxyalkyl groups wherein the alkyl group comprises from 1 to 5 carbon atoms, lower amidoalkyl groups, wherein the alkyl part is C₁-C₄, or alternatively R₁₀ and R₁₁, together with the nitrogen atom to which they are attached, form a heterocyclic group such as piperidyl or morpholinyl; Y⁻ is an anion such as bromide, chloride, acetate, borate, citrate, tartrate, bisulfate, bisulfite, sulfate or phosphate. These polymers are described, for instance in French Patent No. 2 080 759 and in its Certificate of Addition 2 190 406.

In at least one embodiment, R₁₀ and R₁₁ are each, independently chosen from alkyl groups comprising from 1 to 4 carbon atoms.

Among the polymers defined above, non-limiting mention may be made of dialkyldiallylammonium chloride homopolymers, such as the dimethyldiallylammonium chloride homopolymer (INCI name: Polyquaternium-6) sold under the name MERQUAT® 100 by the company Nalco (and its homologues of low weight-average molecular mass) and copolymers of dialkyldiallylammonium chloride, such as the copolymer of dimethyldiallylammonium chloride and of acrylamide, sold under the name MERQUAT® 550.

(8) The quaternary diammonium polymer containing repeating units corresponding to formula (XI):

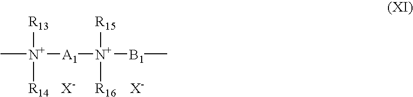

wherein:

R₁₃, R₁₄, R₁₅ and R₁₆, which may be identical or different, are chosen from aliphatic, alicyclic and arylaliphatic groups comprising from 1 to 20 carbon atoms or lower hydroxyalkylaliphatic groups, wherein the alkyl part is C₁-C₄, or alternatively R₁₃, R₁₄, R₁₅ and R₁₆, together or separately, with the nitrogen atoms to which they are attached, form heterocycles optionally comprising a second heteroatom other than nitrogen, or alternatively R₁₃, R₁₄, R₁₅ and R₁₆ are chosen from linear and branched C₁-C₆ alkyl groups substituted with a nitrile, ester, acyl or amide group or a group —CO—O—R₁₇-E or —CO—NH—R₁₇-E where R₁₇ is an alkylene and E is a quaternary ammonium group;

A₁ and B₁ are chosen from polymethylene groups comprising from 2 to 20 carbon atoms which may be linear or branched, saturated or unsaturated, and which may contain, linked to or intercalated in the main chain, at least one aromatic ring or at least one oxygen or sulfur atom or sulfoxide, sulfone, disulfide, amino, alkylamino, hydroxyl, quaternary ammonium, ureido, amide or ester group, and

k is an anion derived from an inorganic or organic acid;

A₁, R₁₃ and R₁₅ can form, with the two nitrogen atoms to which they are attached, a piperazine ring; in one embodiment of the present disclosure, if A₁ is a linear or branched, saturated or unsaturated alkylene or hydroxyalkylene radical, B₁ is a group:

—(CH₂)ₙ—CO-E′—OC—(CH₂)ₙ

wherein n is an integer from 0 to 7 and E′ is chosen from:

a) a glycol residue of formula: —O—Z—O—, wherein Z is chosen from linear and branched hydrocarbon radicals, and groups corresponding to one of the following formulae:

—(CH₂—CH—O)ₓ—CH₂—CH₂—

—[CH₂—CH(CH₃)—O]_{y}—CH₂—CH(CH₃)—

wherein x and y are integers ranging from 1 to 4, representing a defined and unique degree of polymerization or any number ranging 1 to 4 representing an average degree of polymerization;

b) bis-secondary diamine residues such as a piperazine derivatives;

c) bis-primary diamine residues of formula: —NH—Y—NH—, wherein Y is chosen from linear and branched hydrocarbon radicals, or alternatively the divalent radical —CH₂—CH₂—S—S—CH₂—CH₂—;

d) ureylene groups of formula: —NH—CO—NH—.

In one embodiment, X⁻ is an anion such as chloride or bromide.

Polymers of this type are described, for example in French Patent Nos. 2 320 330, 2 270 846, 2 316 271, 2 336 434 and 2 413 907 and U.S. Pat. Nos. 2,273,780, 2,375,853, 2,388,614, 2,454,547, 3,206,462, 2,261,002, 2,271,378, 3,874,870, 4,001,432, 3,929,990, 3,966,904, 4,005,193, 4,025,617, 4,025,627, 4,025,653, 4,026,945 and 4,027,020.

In at least one embodiment of the present disclosure, polymers that consist of repeating units corresponding to the formula (XII) below may be used:

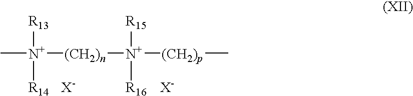

wherein R₁₃, R₁₄, R₁₅ and R₁₆, which may be identical or different, are chosen from alkyl and hydroxyalkyl radicals comprising from 1 to 4 carbon atoms, n and p are integers ranging from 2 to 20, and X⁻ is an anion derived from a mineral or organic acid. In another embodiment, R₁₃, R₁₄, R₁₅ and R₁₆ are each methyl groups. As a non-limiting example of a polymer that may be used corresponding to formula (XII), mention may be made of hexadimethrine chloride, sold under the name MEXOMER PO by the company Chimex.

(9) Poly(quaternary ammonium) polymers formed from units of formula (XIII):

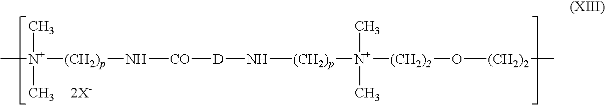

wherein:

p is an integer ranging from 1 to 6,

D is zero or —(CH₂)ᵣ—CO— wherein r is a number equal to 4 or 7, and

X⁻ is an anion derived from a mineral or organic acid.

The cationic polymers comprising units of formula (XIII) are described for example in European Patent Application No. EP-A-122 324 and may be prepared according to the processes described in U.S. Pat. Nos. 4,157,388, 4,390,689, 4,702,906 and 4,719,282.

Such polymers include, but are not limited to those with a molecular mass, measured by carbon-13 NMR, of less than 100,000, and in the formula wherein:

p is equal to 3, and

a) D is a group —(CH₂)₄—CO—, X is a chlorine atom, the molecular mass, measured by carbon-13 NMR (¹³C NMR), being about 5600; a polymer of this type is proposed by the company MIRANOL under the name MIRAPOL-AD1,

b) D is —(CH₂)₇—CO— and X is a chlorine atom, the molecular mass, measured by carbon-13 NMR (¹³C NMR), being about 8100; a polymer of this type is proposed by the company Miranol under the name MIRAPOL-AZ1,

c) D is zero and X is a chlorine atom, the molecular mass, measured by carbon-13 NMR (¹³C NMR), being about 25,500; a polymer of this type is sold by the company Miranol under the name MIRAPOL-A15,

d) a “block copolymer” formed from units corresponding to the polymers described in paragraphs a) and c), sold by the company Miranol under the names MIRAPOL-9 (¹³C NMR molecular mass of about 7800), Mirapol-175 (¹³C NMR molecular mass of about 8000) and Mirapol-95 (¹³C NMR molecular mass of about 12 500).

In at least one embodiment, the polymer that is used herein is a polymer containing units of formula (XIII) wherein p is 3, D is zero and X is a chlorine atom, the molecular mass, measured by carbon-13 NMR (¹³C NMR), being about 25,500.

(10) Quaternary polymers of vinylpyrrolidone and of vinylimidazole, for instance the products sold under the names LUVIQUAT FC 905, FC 550 and FC 370 by the company BASF.

(11) Cationic polyamines such as POLYQUART H sold by Henkel under the reference name Polyethylene Glycol (15) Tallow Polyamine in the CTFA dictionary.

(12) Vinylamide homopolymers or copolymers, such as partially hydrolysed vinylamide homopolymers such as poly(vinylamine/vinylamide)ₛ. These polymers are formed from at least one vinylamide monomer corresponding to the following formula:

H₂C═CR²NRC(O)R¹

wherein R, R¹ and R² are each chosen from a hydrogen atom, C₁-C₂₀ alkyl groups, aryl groups and alkylaryl groups wherein the alkyl part contains from 1 to 20 carbon atoms.

In at least one embodiment, the monomer may be chosen from N-vinylformamide, N-methyl-N-vinylacetamide and N-vinylacetamide. Poly(vinylamine/N-vinylformamide) may also be used, such as the product sold under the name CATIOFAST VMP by the company BASF or under the name LUPAMIN 9030 by the company BASF.

These polymers may be formed, for example, via radical polymerization of a vinylamide monomer followed by partial acidic or basic hydrolysis of the amide functions to quaternizable amine functions, as described in International Patent Application Publication No. WO 2007/005 577, and U.S. Pat. Nos. 5,374,334, 6,426,383 and 6,894,110.

(13) Polyurethanes formed essentially from:

(a1) at least one cationic unit derived from at least one tertiary or quaternary amine containing at least two reactive functions containing labile hydrogen,

(a2) at least one nonionic unit derived from at least one polyolefin containing at least two reactive functions containing labile hydrogen, the said polyolefin comprising at least 10 mol % of units comprising at least one C═C (carbon-carbon) double bond, relative to the total amount of units forming the said polyolefin;

(b) at least one unit derived from a compound comprising at least two isocyanate functions.

The polymer disclosed herein is in at least one embodiment of elastic nature; this means that the said polymer is a macromolecular material that rapidly returns to its initial form and dimensions after a low stress that has produced a large deformation has ceased.

These polymers may be obtained by polycondensation of compounds bearing reactive functions containing labile hydrogen with compounds comprising at least two isocyanate functions.

As used herein, “reactive functions containing labile hydrogen” means functions capable, after loss of a hydrogen atom, of forming covalent bonds with the isocyanate functions of compounds comprising at least two isocyanate functions. Examples of such functions that may be mentioned include hydroxyl, primary amine or secondary amine groups, or thiol groups.

Depending on the nature of the reactive functions bearing the labile hydrogen (—OH, —NH₂, —NHR or —SH), the polycondensation leads, respectively, to polyurethanes, polyureas or polythiourethanes. Thus, the polymers according to the present disclosure may be urethane/urea and/or thiourethane copolymers, which may all be incorporated under the term “polyurethanes.”

The at least one cationic polyurethane that may be used in the composition according to the present disclosure thus comprises at least one cationic unit (a1) resulting from at least one tertiary or quaternary amine containing at least two reactive functions containing labile hydrogen.

The tertiary amine is for example protonatable at a pH ranging from pH 1 to pH 12. As used herein, “protonatable” means that the tertiary amine function may be at least partially neutralized with a neutralizer or as a function of the medium in which it is formulated.

When the tertiary or quaternary amines forming the units (a1) bear more than two functions containing labile hydrogen, the polyurethanes obtained have a branched structure.

However, in one embodiment, the tertiary or quaternary amines forming the units (a1) contain only two reactive functions containing labile hydrogen and the polyurethanes obtained via polycondensation consequently have an essentially linear structure.

It is also possible to use a mixture of difunctional amines containing, or otherwise, a small proportion of amines bearing more than two reactive functions containing labile hydrogen.

According to at least one embodiment, the tertiary or quaternary amines forming the cationic or cationizable units (a1) are chosen from compounds corresponding to at least one of the following formulae:

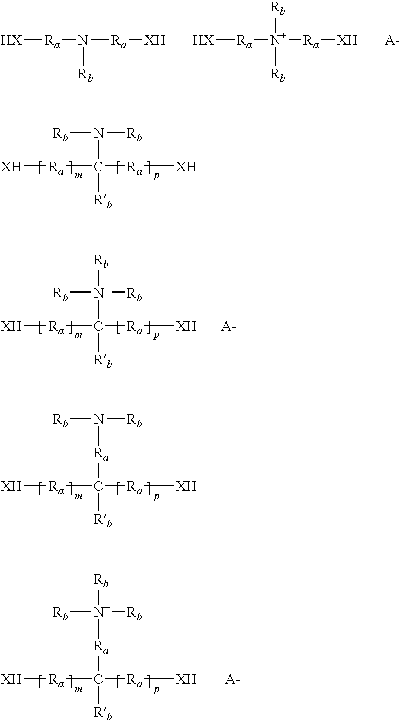

wherein:

each Rₐ, independently of each other, is chosen from linear and branched divalent C₁-C₆ alkylene groups, C₃-C₆ cycloalkylene and arylene groups; optionally substituted with at least one halogen atom and/or comprising at least one heteroatom chosen from O, N, P and S,

each R_{b} independently of each other, is chosen from linear and branched C₁-C₆ alkyl groups, C₃-C₆ cycloalkyl or aryl groups; optionally substituted with at least one halogen atom and/or comprising at least one heteroatom chosen from O, N, P and S,

each R′_{b} independently of each other, is chosen from a hydrogen atom, linear and branched C₁-C₆ alkyl groups, C₃-C₆ cycloalkyl and aryl groups; optionally substituted with at least one halogen atom and/or comprising at least one heteroatom chosen from O, N, P and S,

m and p are, independently of each other, 0 or 1; such as m=1 and p=1;

each X independently of each other, is chosen from an oxygen atom, a sulfur atom, NH, and NR_{c}, wherein R_{c} is chosen from C₁-C₆ alkyl groups, and

A″ is a physiologically acceptable counterion, such as halide, for instance chloride or bromide.

In at least one embodiment, the amines are chosen from compounds corresponding to at least one of the following formula:

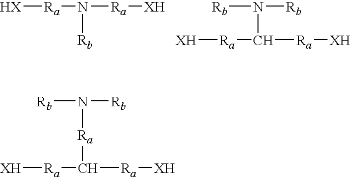

wherein:

Rₐ is chosen from linear and branched divalent C₁-C₆ alkylene groups, such as methylene and ethylene;
R_{b} is chosen from linear and branched C₁-C₆ alkyl groups, for example, methyl, ethyl, n-butyl, isobutyl and tert-butyl groups;
X is an oxygen atom.

In another embodiment, the amines are chosen from

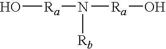

wherein Rₐ is chosen from linear and branched divalent C₁-C₆ alkylene groups, such as methylene and ethylene; and R_{b} is chosen from linear and branched C₁-C₆ alkyl groups, such as methyl, ethyl, n-butyl, isobutyl and tert-butyl groups.

Exemplary tertiary amines that may be used herein include N-methyldiethanolamine and N-tert-butyldiethanolamine.

The protonatable tertiary amines may be totally or partially neutralized with a neutralizer of organic acid type comprising at least one carboxylic, sulfonic and/or phosphonic acid function or with a mineral acid. Non-limiting examples of acids that may be mentioned include hydrochloric acid, sulfuric acid, acetic acid, propionic acid, citric acid, gluconic acid, tartaric acid, lactic acid, phosphoric acid, benzoic acid, stearic acid, oleic acid, 2-ethylcaproic acid, behenic acid and betaine hydrochloride, and mixtures thereof.

The at least one cationic polyurethane that may be used herein also comprise at least one nonionic unit (a2) resulting from at least one polyolefin containing at least two reactive functions containing labile hydrogen, the polyolefin comprising at least 10 mol % of units comprising at least one C═C double bond (residual), relative to the total amount of units forming the polyolefin.

In at least one embodiment, the at least one polyolefin is nonionic.

In one embodiment, the reactive functions containing labile hydrogen are located at the ends of the polyolefin. The reactive functions containing labile hydrogen are for example hydroxides. In another embodiment, the number of hydroxide units is close to or even equal to 2.

In at least one embodiment, the at least one polyolefin forming the unit (a2) is chosen from olefin homopolymers and/or copolymers, bearing at their ends reactive functions containing labile hydrogen and having a glass transition temperature (Tg), measured by differential thermal analysis (DSC, differential scanning calorimetry) according to ASTM standard D3418-97, of less than 10° C.

The at least one polyurethane in the composition according to the present disclosure may comprise several units (a2) resulting from several identical or different polyolefins (polyolefin mixtures); however, in this case, each of the polyolefins comprises at least 10 mol % of units comprising at least one C═C double bond.

As used herein, “unit comprising a C═C double bond” means a unit comprising at least one residual C═C double bond, such as only one double bond; it may be, for example, a unit derived from the polymerization of a butadiene or isoprene unit, all isomeric forms included (cis or trans, 1,2- or 1,4-).

The polyolefin that may be used may be an olefin homopolymer. Examples that may be mentioned include, but are not limited to 1,2-butadiene, 1,4-butadiene and isoprene homopolymers, for example:

1,4-polybutadienes, in their cis and trans forms:

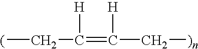

1,2-polybutadienes: —[CH₂—CH(CH═CH2)-]n

poly(cis-1,4-isoprenes):

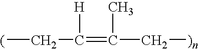

poly(trans-1,4-isoprenes):

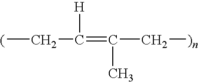

The at least one polyolefin that may be used may also be a copolymer of different olefins (olefin copolymer), provided that the final polyolefin comprises at least 10 mol % of units comprising at least one C═C doulble bond.

In at least one embodiment, the polyolefin may be formed exclusively from units comprising at least one C═C double bond. Examples that may be mentioned include, but are not limited to copolymers, such as statistical copolymers, comprising 1,2-butadiene units and/or 1,4-butadiene units in its cis and/or trans forms, and/or isoprene units, for instance, in some embodiments, cis-1,4-isoprene and trans-1,4-isoprene, as a mixture. Non-limiting mention may be made of (1,2-butadiene/1,4-butadiene) statistical copolymers.

In at least one embodiment, the at least one polyolefin that may be used may be statistical and with hydroxyl end groups and correspond to the following structure:

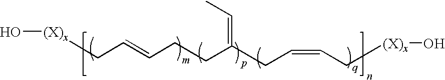

wherein:

m, p and q are mole fractions ranging from 0 to 1, and m+p+q=1; with m ranging from 0.1 to 0.8, for instance ranging from 0.15 to 0.7; p ranging from 0.1 to 0.8, such as ranging from 0.15 to 0.7; and q ranging from 0.05 to 0.5, for instance ranging from 0.1 to 0.4;

n is an integer ranging from 10 to 100, such as from 15 to 50;

x=0 or 1, and

X is chosen from divalent carbon-based groups, for example linear, cyclic and branched alkylene groups, comprising 1 to 10 carbon atoms; for instance methylene, ethylene, propylene and isopropylene groups.

They may, for instance have a number-average molecular mass, Mn, ranging from 400 to 50,000, for example ranging from 500 to 30,000, such as ranging from 1000 to 15,000, for example, ranging from 1,500 to 12,000.

Non-limiting mention may be made of the following:

polybutadienes with hydroxyl end groups, such as the polymers of structure:

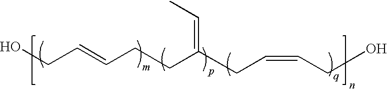

with m=0.6, p=0.2 and q=0.2 (mole fractions) and n=25.

Mention may be made, for example of the commercial products Poly bd R20LM and Poly bd R45HTLO from Sartomer;

polybutadienes with primary hydroxyl end groups, such as the polymers of the following structure:

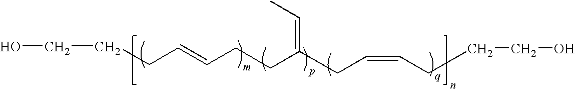

which are statistical copolymers of 1,4-cis-butadiene and of 1,4-trans-butadiene, with m=0.17, p=0.65 and q=0.18 (mole fractions) and n is such that the number-average molecular weight Mn ranges from 1,000 to 10,000, for instance from 2,000 to 6,000 (g.mol⁻¹).

Non-limiting mention may be made, for example of the commercial products KRASOL LBH-P 2000, 3000 or 5000 from Sartomer;

polybutadienes with secondary hydroxyl end groups, such as the polymers of the following structure:

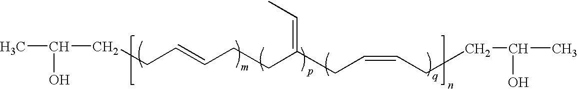

which are statistical copolymers of 1,4-cis-butadiene and of 1,4-trans-butadiene, with m=0.17, p=0.65 and q=0.18 (mole fractions), and n is such that the number-average molecular weight Mn ranges from 1,000 to 12,000, such as from 2,000 to 10,000 (g.mol⁻¹).

Mention may be made, for example of the commercial products KRASOL LBH 2000, 3000, 5000 or 10 000 from Sartomer.

In some embodiments, the at least one polyolefin may also comprise additional units not comprising a C═C double bond.

However, these additional units are present in a maximum amount of 90 mol %, given that the final polyolefin should comprise at least 10 mol % of units comprising at least one C═C double bond.

These additional olefin units may be chosen from, for instance ethylene —(CH₂—CH₂)ₙ—, propylene —(CH₂—CH₂—CH₂)ₙ— isopropylene —(CH₂CH(CH₃))ₙ— units, and butylene units of formula:

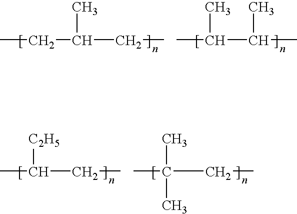

and also mixtures thereof.

The olefin homopolymers or copolymers as defined above may undergo, after polymerization, a partial hydrogenation of the residual double bonds. This hydrogenation may not be total.

Specifically, the polyolefins that may be used to form the units (a2) according to the present disclosure should comprise at least 10 mol % of units comprising at least one C═C double bond (residual), relative to the total amount of units forming the polyolefin.

They comprise, for example at least 20 mol %, such as at least 40 mol %, for instance at least 50 mol %, such as at least 80 mol %, for example 100 mol %, of units comprising at least one C═C double bond, for instance units comprising only one C═C double bond.

This content of units comprising at least one C═C double bond may be determined via the usual techniques, such as via NMR or iodine assay.

According to at least one embodiment, the at least one polyolefin that may be used to form the nonionic units (a2) has a number-average molecular mass (Mn) ranging from 400 to 50,000, for example ranging from 500 to 30 000, such as ranging from 1000 to 15 000, for instance ranging from 1500 to 12 000.

In some embodiments, the at least one polyolefin that may used herein include, but are not limited to:

homopolymers such as 1,4-polybutadiene and 1,2-polybutadiene;

copolymers of structure:

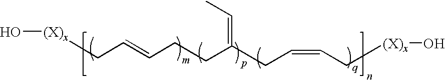

wherein:

m, p and q are mole fractions ranging from 0 to 1, and m+p+q=1;

with m ranging from 0.1 to 0.8, such as ranging from 0.15 to 0.7; p ranging from 0.1 to 0.8, for example, ranging from 0.15 to 0.7; and q ranging from 0.05 to 0.5, such as ranging from 0.1 to 0.4;

n is an integer ranging from 10 to 100, for example ranging from 15 to 50;

x=0 or 1, and

X is chosen from divalent carbon-based radicals, for example linear, cyclic and branched alkylene groups comprising 1 to 10 carbon atoms; for instance a methylene, ethylene, propylene or isopropylene group.

The at least one cationic polyurethane that may be used in the composition according to the present disclosure also comprise at least one unit (b) resulting from at least one compound comprising at least two isocyanate functions.

In at least one embodiment, it may be a mixture of several compounds comprising at least two isocyanate functions.

The compounds comprising at least two isocyanate functions may be chosen from diisocyanates, and mixtures of a diisocyanate and a polyisocyanate comprising more than two isocyanate functions, the polyisocyanate present in an amount ranging from 0.1% to 40% of the weight of the said mixture, such as ranging from 0.5% to 35% by weight, for example ranging from 1% to 30% by weight relative to the weight of the mixture.

The compounds comprising at least two isocyanate functions may be chosen from conjugated and non-conjugated, aromatic and non-aromatic cyclic aliphatic diisocyanates. Non-limiting examples include but are not limited to methylenediphenyl diisocyanate, methylenecyclohexane diisocyanate, isophorone diisocyanate, toluene diisocyanate, naphthalene diisocyanate, 1,4-butane diisocyanate, 1,6-hexane diisocyanate, and mixtures thereof; such as isophorone diisocyanate.

In at least one embodiment, the at least one polyurethane that may be used in the composition according to the present disclosure are formed essentially from:

at least one cationic unit resulting from amines of formula:

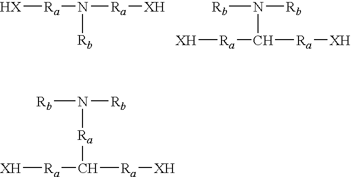

wherein:

Rₐ is chosen from linear and branched divalent C₁-C₆ alkylene groups, such as methylene and ethylene groups;

R_{b} is chosen from linear and branched C₁-C₆ alkyl groups, for example methyl, ethyl, n-butyl, isobutyl and tert-butyl groups;

and X is a hydrogen atom;

at least one nonionic unit resulting from polyolefins chosen from 1,4-polybutadiene and 1,2-polybutadiene homopolymers; and copolymers of structure:

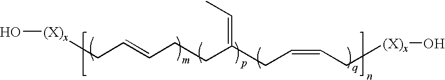

wherein

m, p and q are mole fractions ranging from 0 to 1, and m+p+q=1;

for instance m ranging from 0.1 to 0.8, such as ranging from 0.15 to 07; p ranging from 0.1 to 0.8, for example ranging from 0.15 to 0.7; and q ranging from 0.05 to 0.5, such as ranging from 0.1 to 0.4;

n is an integer ranging from 10 to 100, for instance ranging from 15 to 50;

x=0 or 1, and

X is a divalent carbon-based group, such as linear, cyclic and branched alkylene groups, comprising 1 to 10 carbon atoms; for instance a methylene, ethylene, propylene or isopropylene group;

at least one unit resulting from aliphatic diisocyanates.

In at least one embodiment, the at least one polyurethane that may be used according to the present disclosure is formed essentially from:

at least one cationic unit resulting from amines of formula:

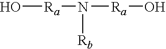

wherein Rₐ is chosen from linear and branched divalent C₁-C₆ alkylene groups, such as methylene and ethylene groups; and R_{b} is chosen from linear and branched C₁-C₆ alkyl groups, for example methyl, ethyl, n-butyl, isobutyl and tert-butyl groups;

and for example at least one cationic unit chosen from N-methyldiethanolamine and N-tert-butyldiethanolamine;

at least one nonionic unit resulting from polyolefins of structure:

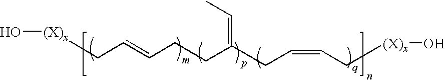

wherein

m, p and q are mole fractions ranging from 0 to 1, and m+p+q=1;

for instance m ranging from 0.1 to 0.8, such as ranging from 0.15 to 0.7; p ranging from 0.1 to 0.8, for example ranging from 0.15 to 0.7; and q ranging from 0.05 to 0.5, such as ranging from 0.1 to 0.4;

n is an integer ranging from 10 to 100, such as ranging from 15 to 50;

x=0 or 1, and

X is chosen from divalent carbon-based groups, for example linear, cyclic and branched alkylene groups, comprising 1 to 10 carbon atoms; for instance methylene, ethylene, propylene and isopropylene groups;

at least one unit resulting from diisocyanates chosen from methylenecyclohexane diisocyanate, isophorone diisocyanate, 1,4-butane diisocyanate and 1,6-hexane diisocyanate, and mixtures thereof; such as isophorone diisocyanate.

The polyurethanes that may be used according to the present disclosure are formed essentially from units (a1), (a2) and (b) as defined above, which implies that they do not comprise additional units other than these.

Among all the polyurethanes mentioned herein, non-limiting examples of polyurethanes useful herein include those formed from the following monomers:

(a1) at least one N-methyldiethanolamine (noted NMDEA),

(a2) at least one nonionic ethylene/butylene copolymer such as the product sold under the name KRASOL LBH-P 2000, and

(b) at least one isophorone diisocyanate (noted IPDI).

In at least one embodiment, the amines forming the cationic units (a1) are present in an amount ranging from 0.1% to 50%, such as ranging from 1% to 30%, for instance, ranging from 5% to 20% by weight relative to the total weight of the final polyurethane.

In another embodiment, the polyurethanes forming the nonionic units (a2) are present in an amount ranging from 30% to 99% by weight, such as ranging from 50% to 90%, for instance ranging from 60% to 80% by weight relative to the total weight of the final polyurethane.

According to at least one embodiment of the present disclosure, the compounds comprising at least two isocyanate functions, forming the units (b), are present in an essentially stoichiometric amount relative to the sum of the tertiary/quaternary amines forming the units (a1) and of the polyolefins forming the units (a2).

In at least one embodiment, the compounds comprising at least two isocyanate functions forming the units (b) are present in an amount ranging from 1% to 60% by weight, such as ranging from 5% to 50% by weight for example ranging from 15% to 35% by weight relative to the total weight of the final polyurethane.

In another embodiment, the polyurethanes according to the present disclosure are formed from:

20% to 55%, such as from 25% to 50%, for example from 30% to 47%, on a molar basis, of tertiary or quaternary amine capable of forming the units (a1);

1% to 30%, such as from 2% to 25% for example from 3% to 20%, on a molar basis, of polyolefins capable of forming the units (a2); and

30% to 65%, such as 35% to 60% for example from 45% to 55%, on a molar basis, of compound comprising at least two isocyanate functions capable of forming the units (b).

In another embodiment, the mole ratio between (b) and the sum of (a1)+(a2) has a value close to 1.

These polyurethanes and their synthesis are described, for example, in French Patent Application No. FR-A-2 898 603.

(14) Other cationic polymers that may be used according to the present disclosure are cationic proteins or cationic protein hydrolysates, polyalkyleneimines, such as polyethyleneimines, polymers containing vinylpyricline or vinylpyridinium units, condensates of polyamines and of epichlorohydrin, quaternary polyureylenes and chitin derivatives.

Among all the cationic polymers that may be used herein include, but are not limited to, alone or as mixtures, at least one cationic polymer chosen from:

copolymers of acrylamide and of dimethylaminoethyl methacrylate quaternized with dimethyl sulfate or with a dimethyl halide,

copolymers of acrylamide and of methacryloyloxyethyltrimethylammonium methosulfate,

quaternized or non-quaternized vinylpyrrolidone/dialkylaminoalkyl acrylate or methacrylate copolymers. These polymers are described in detail in French Patent Nos. 2 077 143 and 2 393 573,

dimethylaminoethyl methacrylate/vinylcaprolactam/vinylpyrrolidone terpolymers,

vinylpyrrolidone/methacrylamidopropyldimethylamine copolymers, and

quaternized vinylpyrrolidone/dimethylaminopropylmethacrylamide copolymers,

quaternary ammoniums of hydroxyethylcellulose that has reacted with an epoxide substituted with a trimethylammonium group (INCL name: Polyquaternium-10),

hydroxyalkylcelluloses, for instance hydroxymethyl-, hydroxyethyl- or hydroxypropyl-celluloses grafted, for example with a methacryloylethyltrimethylammonium, methacrylamidopropyltrimethylammonium or dimethyldiallylammonium salt,

guar gums containing trialkylammonium cationic groups,

dimethyldiallylammonium chloride homopolymer (INCI name: Polyquaternium-6) sold under the name MERQUAT® 100 by the company Nalco (and homologues thereof of low weight-average molecular mass),

hexadimethrine chloride,

quaternary polymers of vinylpyrrolidone and of vinylimidazole,

poly(vinylamine/vinylamide)s,

polyurethanes formed from the following monomers:

(a1) at least one N-methyldiethanolamine (noted NMDEA),

(a2) at least one nonionic ethylene/butylene copolymer such as the product sold under the name KRASOL LBH-P 2000, and

(b) at least one isophorone diisocyanate (noted IPDI),
polyalkyleneimines, in particular polyethyleneimines.

When the at least one polymer (F) is present in the composition according to the present disclosure, it is present in amount ranging from 0.01% to 10% by weight, such as ranging from 0.05% to 8% by weight, for instance ranging from 0.1% to 5% by weight relative to the total weight of the composition.

Agent that is Beneficial to Keratin Materials

The composition of the present disclosure may also comprise at least one agent beneficial to keratin materials, other than the compounds (A), (B), (C), (D), (E) and (F) mentioned above, chosen from:

(1) additional saccharides, oligosaccharides and polysaccharides other than those mentioned previously, which may be hydrolysed or non-hydrolysed, and modified or unmodified,

(2) hydrolysed or non-hydrolysed, modified or unmodified amino acids, oligopeptides, peptides and proteins,

(3) polyols and polyethylene glycols,

(4) emollients,

(5) moisturizers,

(6) hydroxylated organic acids,

(7) UV-screening agents,

(8) antioxidants and free-radical scavengers,

(9) chelating agents,

(10) antidandruff agents,

(11) anti-alopecia agents,

(12) seborrhoea regulators,

(13) calmatives,

(14) cationic surfactants,

(15) organomodified or non-organomodified, volatile or non-volatile silicones,

(16) anionic polymers,

(17) nonionic polymers,

(18) amphoteric polymers,

(19) pigments,

(20) mineral or organic fillers,

(21) clays,

(22) colloidal minerals.

When the at least one beneficial agent is present in the compositions herein, it is present in an amount ranging from 0.001% to 10% by weight, for example ranging from 0.01% to 5% by weight, such as ranging from 0.1%) to 3% by weight relative to the total weight of the composition.

The detergent composition according to the present disclosure has, in at least one embodiment, a pH ranging from 2 to 10, for example ranging 4 to 7. Adjusting the pH to the desired value may be performed conventionally by adding at least one base (organic or mineral) to the composition, for example sodium hydroxide, aqueous ammonia or a primary, secondary or tertiary (poly)amine, for instance monoethanolamine, diethanolamine, triethanolamine, isopropanolamine or 1,3-propanediamine, or alternatively by adding a mineral or organic acid, such as a polycarboxylic acid such as citric acid, tartaric acid, maleic acid, succinic acid or adipic acid, and mixtures thereof.

According to at least one embodiment of the present disclosure, the composition according to the present disclosure comprises a cosmetically acceptable medium.

The cosmetically acceptable medium may be an aqueous medium formed solely from water or from a mixture of water and at least one cosmetically acceptable solvent, such as a C₁-C₄ lower alcohol, for instance ethanol, isopropanol, tert-butanol or n-butanol; alkylene glycols, for instance propylene glycol or hexylene glycol; glycerol; and mixtures thereof.

The composition according to the present disclosure comprises at least 30% by weight, such as from 50% to 90% by weight, for instance from 70% to 85% by weight of water relative to the total weight of the composition.

The composition according to the present disclosure may comprise, in addition to compounds (A), (B), (C), (D) and (E) as defined previously, at least one cosmetic additive.

As used herein, “cosmetic additive” means a cosmetically acceptable compound other than the compounds (A), (B), (C), (D) and (E) used herein, and added to the composition of the disclosure. When the composition according to the present disclosure comprises at least one polymer (F) and/or at least one agent that is beneficial to keratin materials, the composition also comprises at least one additive different from the polymers (F) and from the beneficial agents.

Among the cosmetic additives that may be used, non-limiting mention may be made of conventional adjuvants that are well known in the art, such as non-polymeric electrolytes, reducing agents, oxidizing agents, direct dyes or oxidation dyes, polymeric or non-polymeric thickeners, organic or mineral acids and bases, plasticizers, optical brighteners, nacres, nacreous agents, fragrances, peptizers, preserving agents, and surfactants other than those disclosed herein, and mixtures thereof.

Such additives may be present in the composition according to the present disclosure in an amount ranging from 0% to 20% by weight relative to the total weight of the composition.

A person skilled in the art will take care to select the at least one optional additive and the amount thereof such that they do not harm the properties of the compositions of the present disclosure.

Another aspect of the present disclosure is a cosmetic treatment process, characterized in that it comprises the application to keratin materials, such as human keratin fibers for example the hair, of an effective amount of a composition according to the present disclosure.

The composition may be applied to wet or dry hair, such as wet or moist hair.

According to at least one embodiment, such a process consists in applying to the hair an effective amount of the composition according to the present disclosure, optionally massaging the hair, optionally leaving the composition to stand on the hair, and rinsing.

When the said composition is left to stand on the hair, the leave-on time generally ranges from 0.5 to 5 minutes. The composition is generally rinsed out with water.

Another aspect of the present disclosure is also the use of a composition disclosed herein for the manufacture of a detergent cosmetic composition for keratin fibers, for example for human keratin fibers, such as the hair, for instance the manufacture of a conditioning shampoo.

Another aspect of the present disclosure relates to the use of a composition disclosed herein for treating, for example, for conditioning keratin materials, for instance human keratin fibers, such as the hair.

Notwithstanding that the numerical ranges and parameters setting forth the broad scope of the disclosure are approximations, unless otherwise indicated the numerical values set forth in the specific examples are reported as precisely as possible. Any numerical value, however, inherently contains certain errors necessarily resulting from the standard deviation found in their respective testing measurements.

The following examples serve to illustrate embodiments of the present disclosure without, however, exhibiting a limiting nature.

### EXAMPLES

The following shampoo compositions were prepared. Unless otherwise indicated, the amounts are given as weight percentages of active material relative to the total weight of the composition. The amounts of commercial products used given as weight percentage relative to the total weight of the composition are indicated in brackets.

| Compositions | Ex. 1 | Ex. 2 | Ex. 3 | Ex. 4 |
|---|---|---|---|---|
| 2-Oleamido-1,3-octadecanediol | 0.5 | 0.3 | — | — |
| Hydrogenated polyisobutene [1] | — | 1 | — | — |
| Hydrogenated jojoba wax | — | — | 1% | — |
| Carnauba wax | — | — | — | 0.2 |
| Polyquaternium-6 at 40% by weight in water [2] | 0.7 (1.75) | 0.4 (1) | 0.4 (1) | — |
| Polyquaternium-10 at 91% by weight in a | — | — | 0.7 (0.8) | 0.7 (0.8) |
| water/isopropanol mixture [3] | | | | |
| Propylene glycol | 2 | 2 | 2 | 2 |
| Cocobetaine at 30% by weight in water [4] | 5.4 (18) | 5.4 (18) | 5.4 (18) | 5.4 (18) |
| Sodium chloride | 2.5 | 2.5 | — | 2.5 |
| Sodium lauryl ether carboxylates (5 EO) at 90% | 3 (3.3) | 3 (3.3) | 3 (3.3) | 3 (3.3) |
| by weight in water [5] | | | | |
| Sodium lauryl ether sulfate (2.2 EO) at 70% by | 5.7 | [symbol] | [symbol] | [symbol] |
| weight in water [6] | 5.7) | 5.7) | 5.7) | 5.7) |
| Alkyl(C₈-C₁₆)polyglucoside at 53% by weight in | [symbol] (9.45) | [symbol] (9.45) | [symbol] (9.45) | [symbol] (9.45) |
| water [7] | | | | |
| Salicylic acid | 0.2 | 0.2 | 0.2 | 0.2 |
| Ethyl para-hydroxybenzoate | 0.15 | 0.15 | 0.15 | 0.15 |
| Sodium benzoate | 0.5 | 0.5 | 0.5 | 0.5 |
| Methyl para-hydroxybenzoate, sodium salt | 0.4 | 0.4 | 0.4 | 0.4 |
| Fragrance | 0.5 | 0.5 | 0.5 | 0.5 |
| Water | qs 100 | qs 100 | qs 100 | qs 100 |

[1] PARLEAM sold by NOF Corporation
[2] MERQUAT 100 sold by Nalco
[3] UCARE Polymer JR 400 LT sold by Amerchol
[4] DEHYTON AB 30 sold by Cognis
[5] AKYPO RLM 45 sold by KAO
[6] TEXAPON AOS 225 UP sold by Cognis
[7] PLANTACARE 818 UP sold by Cognis [symbol]
indicates data missing or illegible when filed

| Compositions | Ex. 5 | Ex. 6 | Ex. 7 | Ex. 8 | Ex. 9 | Ex. 10 |
|---|---|---|---|---|---|---|
| Avocado oil | 0.6 | 1 | 1.5 | — | — | — |
| Hydrogenated | — | 0.5 | — | 2 | — | — |
| polyisobutene [1] | | | | | | |
| Apricot oil | — | — | — | — | 1 | — |
| Isopropyl myristate | — | — | — | — | — | 2 |
| Polyquaternium-6 at | — | — | 0.4 (1) | 0.7 (1.75) | 0.7 (1.75) | — |
| 40% by weight in | | | | | | |
| water [2] | | | | | | |
| Polyquaternium-10 at | 0.5 (0.5) | 0.7 (0.8) | — | — | — | 0.7 (0.8) |
| 91% by weight in a | | | | | | |
| water/isopropanol | | | | | | |
| mixture [3] | | | | | | |
| Propylene glycol | 2 | 2 | 2 | 2 | 2 | 2 |
| Cocobetaine at 30% | 5.4 (18) | 5.4 (18) | 5.4 (18) | 5.4 (18) | 5.4 (18) | 5.4 (18) |
| by weight in water [4] | | | | | | |
| Ceteareth-60 myristyl | — | — | 2.1 | 2.1 | 2.1 | — |
| glycol [5] | | | | | | |
| Sodium chloride | — | — | — | 2.5 | — | — |
| Sodium lauryl ether | 3 (3.3) | 3 (3.3) | 3 (3.3) | 3 (3.3) | 3 (3.3) | 3 (3.3) |
| carboxylates (5 EO) | | | | | | |
| at 90% by weight in | | | | | | |
| water [6] | | | | | | |
| Sodium lauryl ether | 4 (5.7) | 4 (5.7) | 4 (5.7) | 4 (5.7) | 4 (5.7) | 4 (5.7) |
| sulfate (2.2 EO) at | | | | | | |
| 70% by weight in | | | | | | |
| water [7] | | | | | | |
| Alkyl(C₈-C₁₆) | 5 (9.45) | 5 (9.45) | 5 (9.45) | 5 (9.45) | 5 (9.45) | 5 (9.45) |
| polyglucoside at 53% | | | | | | |
| by weight in water [8] | | | | | | |
| Salicylic acid | 0.2 | 0.2 | 0.2 | 0.2 | 0.2 | 0.2 |
| Ethyl para-hydroxy- | 0.15 | 0.15 | 0.15 | 0.15 | 0.15 | 0.15 |
| benzoate | | | | | | |
| Sodium benzoate | 0.5 | 0.5 | 0.5 | 0.5 | 0.5 | 0.5 |
| Methyl para-hydroxy- | 0.4 | 0.4 | 0.4 | 0.4 | 0.4 | 0.4 |
| benzoate, sodium salt | | | | | | |
| Fragrance | 0.5 | 0.5 | 0.5 | 0.5 | 0.5 | 0.5 |
| Water | qs 100 | qs 100 | qs 100 | qs 100 | qs 100 | qs 100 |

[1] PARLEAM sold by NOF Corporation
[2] MERQUAT 100 sold by Nalco
[3] UCARE Polymer JR 400 LT sold by Amerchol
[4] DEHYTON AB 30 sold by Cognis
[5] ELFACOS GT 2825 sold by Akzo Nobel
[6] AKYPO RLM 45 sold by KAO
[7] TEXAPON AOS 225 UP sold by Cognis
[8] PLANTACARE 818 UP sold by Cognis

Results:

The compositions of Examples 1 to 10 had good foam qualities (initiation and abundance).

Wet hair was smooth and supple, and disentangled easily.

Dry hair was smooth, well-managed and shiny.

What is claimed is:

1. A composition for washing and conditioning keratin materials, comprising:
at least one anionic surfactant (A) comprising in its structure at least one group chosen from sulfate, sulfonate and phosphate groups,
at least one carboxylic alkyl ether anionic surfactant (B) other than the at least one anionic surfactant (A),
at least one surfactant chosen from amphoteric and zwitterionic surfactants (C),
at least one alkyl(poly)glycoside nonionic surfactant (D),
at least one non-silicone fatty substance (E), and
optionally at least one cationic polymer (F), wherein
the weight ratio of the amount of the at least one anionic surfactant (A) to the amount of the at least one nonionic surfactant (D) has a value ranging from 0.5 to 1.

2. The composition according to claim 1, wherein the at least one surfactant (A) is chosen from alkyl sulfates, alkylamido sulfates, alkyl ether sulfates, alkylamido ether sulfates, alkylaryl ether sulfates, alkyl ether sulfosuccinates, acyl isethionates, methyl acyl taurates, and salts thereof.

3. The composition according to claim 2, wherein the alkyl and acyl groups contain from 8 to 24 carbon atoms, and the aryl group is chosen from phenyl and benzyl groups.

4. The composition according to claim 1, wherein the at least one surfactant (A) is in the form of a salt chosen from sodium salts, ammonium salts, amine salts, including amino alcohol salts, and magnesium salts.

5. The composition according to claim 1, wherein the at least one surfactant (A) is oxyethylenated and/or oxypropylenated.

6. The composition according to claim 1, wherein the at least one surfactant (A) is present in an amount ranging from 1% to 50% by weight relative to the total weight of the composition.

7. The composition according to claim 1, wherein the at least one surfactant (B) is polyethoxylated and chosen from those of formula (I):
R₁(OC₂H₄)ₙOCH₂COOA (I)
wherein:
R₁ is chosen from linear and branched C₈-C₂₂ alkyl and alkenyl groups, (C₈-C₉)alkylphenyl groups, R₂CONH—CH₂—CH₂— wherein R₂ is chosen from linear and branched C₁₁-C₂₁ alkyl and alkenyl groups,
n is an integer or decimal number (average value) ranging from 2 to 24, and
A is chosen from H, NH₄, Na, K, Li, Mg, a monoethanolamine and a triethanolamine residue.

8. The composition according to claim 1, wherein the at least one surfactant (B) is present in an amount ranging from 0.5% to 15% by weight relative to the total weight of the composition.

9. The composition according to claim 1, wherein the at least one surfactant (C) is chosen from betaines.

10. The composition according to claim 1, wherein the at least one surfactant (C) is present in an amount ranging from 0.1% to 20% by weight relative to the total weight of the composition.

11. The composition according to claim 1, wherein the at least one surfactant (D) is chosen from those of formula (IV):
R₁O—(R₂O)ₜ-(G)ᵥ (IV)
wherein:
R₁ is chosen from linear and branched, saturated and unsaturated alkyl groups, comprising from about 8 to 24 carbon atoms, and alkyl phenyl groups wherein the linear and ranched alkyl radicals comprise from 8 to 24 carbon atoms,
R₂ is chosen from alkylene groups comprising from 2 to 4 carbon atoms,
G is a saccharide unit comprising 5 or 6 carbon atoms,
t is a value ranging from 0 to 10, and
v is a value ranging from 1 to 15.

12. The composition according to claim 1, wherein the at least one surfactant (D) is present in an amount ranging from 0.1% to 25% by weight relative to the total weight of the composition.

13. The composition according to claim 1, wherein the total amount of anionic, amphoteric surfactant and/or zwitterionic, and of nonionic surfactants, ranges from 4% to 50% by weight relative to the total weight of the composition.

14. The composition according to claim 1, wherein at least one of the weight ratios chosen from
the amount of the at least one surfactant (A) to the amount of the at least one surfactant (B),
the amount of the at least one surfactant (A) to the amount of the at least one surfactant (C), and
the amount of the at least one surfactant (B) to the amount of the at least one surfactant (C) has a value ranging from 0.1 to 10.

15. The composition according to claim 1, wherein the at least one non-silicone fatty substance (E) is chosen from the following non-silicone solid fatty substances:
solid fatty substances not containing an amide function, chosen from waxes of animal, plant, mineral or marine origin, fatty esters, fatty acids and fatty alcohols, and
solid fatty substances containing at least one amide function, chosen from ceramides and ceramide derivatives;
and/or the following non-silicone liquid fatty substances:
liquid fatty esters,
hydrocarbon-based oils,
liquid fatty acids, and
liquid fatty alcohols.

16. The composition according to claim 1, wherein the at least one non-silicone fatty substance (E) is present in an amount ranging from 0.05% to 8% by weight relative to the total weight of the composition.

17. The composition according to claim 1, wherein the at least one polymer (F) is present in an amount ranging from 0.01% to 10% by weight relative to the total weight of the composition.

18. A cosmetic treatment process for washing and conditioning keratin materials, comprising
applying to the keratin materials an effective amount of a composition comprising
at least one anionic surfactant (A) comprising in its structure at least one group chosen from sulfate, sulfonate and phosphate groups,
at least one carboxylic alkyl ether anionic surfactant (B) other than the at least one anionic surfactant (A),
at least one surfactant chosen from amphoteric and zwitterionic surfactants (C),
at least one alkyl(poly)glycoside nonionic surfactant (D),
at least one non-silicone fatty substance (E), and
optionally at least one cationic polymer (F), wherein
the weight ratio of the amount of the at least one anionic surfactant (A) to the amount of the at least one nonionic surfactant (D) has a value ranging from 0.5 to 1.

19. A method for making a composition for washing and treating keratin materials, comprising combining:
at least one anionic surfactant (A) comprising in its structure at least one group chosen from sulfate, sulfonate and phosphate groups,
at least one carboxylic alkyl ether anionic surfactant (B) other than the at least one anionic surfactant (A),
at least one surfactant chosen from amphoteric and zwitterionic surfactants (C),
at least one alkyl(poly)glycoside nonionic surfactant (D),
at least one non-silicone fatty substance (E), and
optionally at least one cationic polymer (F), wherein
the weight ratio of the amount of the at least one anionic surfactant (A) to the amount of the at least one nonionic surfactant (D) has a value ranging from 0.5 to 1.
